(19) United States
(12) Patent Application Publication
(10) Pub. No.: US 2021/0077031 A1
(43) Pub. Date: Mar. 18, 2021

(54) ELECTRONIC APPARATUS AND METHOD

(71) Applicant: KABUSHIKI KAISHA TOSHIBA, Minato-ku (JP)

(72) Inventors: Izumi FUKUNAGA, Setagaya (JP); Takashi SUDO, Fuchu (JP); Yasuhiro KANISHIMA, Suginami (JP)

(73) Assignee: KABUSHIKI KAISHA TOSHIBA, Minato-ku (JP)

(21) Appl. No.: 16/808,622

(22) Filed: Mar. 4, 2020

(30) Foreign Application Priority Data
Sep. 13, 2019 (JP) 2019-167623

(51) Int. Cl. A61B 5/00; A61B 5/16; G06F 3/01

(52) U.S. Cl. CPC A61B 5/7221; A61B 5/165; G06F 2203/011; G06F 3/015; G06F 3/017; A61B 5/742

(57) ABSTRACT

According to one embodiment, an electronic apparatus includes one or more processors. The one or more processors are configured to acquire first information related to reliability of biological information, calculate reliability information of the biological information based on the first information, and associate the reliability information with the biological information.

### CROSS-REFERENCE TO RELATED APPLICATIONS

This application is based upon and claims the benefit of priority from Japanese Patent Application No. 2019-167623, filed Sep. 13, 2019, the entire contents of which are incorporated herein by reference.

### FIELD

Embodiments described herein relate generally to an electronic apparatus and a method, which are capable of determining the reliability of biological information.

### BACKGROUND

Electronic apparatuses used to determine the degree of stress have recently been developed. Psychological stress regarding the operation of an autonomic nerve and physical stress are known as stress on a living body. As an index to determine the degree of psychological stress, there is a stress index regarding autonomic nerve balance called LF/HF which is obtained from heartbeat interval fluctuations and which represents an activity level of sympathetic and parasympathetic nerves. Heartbeat intervals can be obtained by detecting an R wave from an electrocardiogram. Note that pulse intervals may be used in place of the heartbeat intervals.

Physical stress includes stress due to fatigue, pressure, heat, etc. For example, as an index to determine the degree of physical stress, there is a WBGT index. The WBGT index is set for the prevention of heatstroke and indicates a dangerous level of heatstroke. The WBGT index adopts humidity, radiant heat, and air temperature, which have a great effect on the heat balance of the human body. The WBGT index is calculated from natural dry bulb temperature, natural wet bulb temperature, globe temperature, solar radiation, average wind speed, and the like.

The degree of psychological or physical stress may be determined based on the value of the stress index itself, or the presence or absence of the stress may be determined by comparing the value of the stress index and a threshold.

Electrocardiograms, which are weak electrical signals, are susceptible to noise. While a living body is at rest, an electrocardiogram does not show noise due to body motion. While the living body is moving, an electrocardiogram may show noise due to fluctuations in the contact of a sensor with the living body, swing of the sensor itself, irregularities of the blood flow, etc. When noise affects the electrocardiogram, the reliability of the stress index decreases.

A conventional electronic apparatus that processes weak signals as described above includes a body motion detector. Heartbeat interval data acquired when the body motion detector has determined that “the body is moving” includes noise and is likely to be incorrect. The electronic apparatus cancels the heartbeat interval data without using it for the calculation of a psychological stress index. When the heartbeat interval data is canceled, it is lost and the calculation of the psychological stress index is interrupted. However, the heartbeat interval data (and the psychological stress index calculated therefrom) is not necessarily “incorrect”. Even though the body is moving, heartbeat interval data may correctly be acquired. Conventionally, however, the reliability of biological information that is heartbeat interval data acquired by the electronic apparatus and/or psychological stress index calculated by the electronic apparatus, has not been identified. Therefore, correct heartbeat interval data may also be canceled and the acquired heartbeat interval data will be wasted.

Like the psychological stress index, noise affects a physical stress index based on a heart rate.

Noise also affects the WBGT index. Noise is generated due to fever of a person being measured due to an infectious disease, a state in which only the sensor gets hot, or a state in which the apparatus is overheated. The sensor gets hot, for example, if a person being measured winds his or her wrist with a wristband sensor and his or her arm is in a “kotatsu”.

The apparatus is overheated, for example, if the processing capacity of the apparatus cannot catch up with index calculation or index comparison processing.

### BRIEF DESCRIPTION OF THE DRAWINGS

FIG. 1 is a block diagram showing an example of a system including an electronic apparatus according to a first embodiment.

FIG. 2 is a block diagram showing an example of a configuration of a sensor included in the system of FIG. 1.

FIG. 3 is a block diagram showing an example of a configuration of a mobile terminal included in the system of FIG. 1.

FIG. 4 is a functional block diagram showing an example of a stress estimation program executed by the mobile terminal of FIG. 1.

FIG. 5 is a chart showing an example of LF and HF calculated by the stress estimation program.

FIG. 6 is an illustration of a first example of display of the mobile terminal.

FIG. 7 is a functional block diagram showing an example of a stress estimation program executed by a mobile terminal according to a second embodiment.

FIGS. 8A, 8B, 8C, and 8D are charts showing examples of an electrocardiogram, body motion, contact pressure, and sweat quantity, which are acquired by the stress estimation program.

FIGS. 9A, 9B, 9C, and 9D are charts showing examples of the relationship between the reliability of a stress index and heart rate, the body motion, contact pressure, and sweat quantity.

FIG. 10 is an illustration of a second example of display of the mobile terminal.

FIG. 11 is a functional block diagram showing a modification of the stress estimation program executed by the mobile terminal according to the second embodiment.

FIG. 12 is a functional block diagram showing an example of a stress estimation program executed by a mobile terminal according to a third embodiment.

FIG. 13 is a table showing an example of the relationship between the reliability of a stress index and the body motion, contact pressure, and sweat quantity.

FIG. 14 is an illustration of a third example of display of the mobile terminal.

FIG. 15 is a functional block diagram showing an example of a stress estimation program executed by a mobile terminal according to a fourth embodiment.

FIG. 16 is a chart showing an example of heartbeat interval restoration to be executed by the stress estimation program.

FIG. 17 is an illustration of a fourth example of display of the mobile terminal.

FIG. 18 is a functional block diagram showing an example of a stress estimation program executed by a mobile terminal according to a fifth embodiment.

FIG. 19 is a chart showing an example of integrated reliability calculation to be executed by the stress estimation program.

FIG. 20 is a functional block diagram showing an example of a stress estimation program executed by a mobile terminal according to a sixth embodiment.

FIG. 21 is a functional block diagram showing an example of a heat stress determination module of the stress estimation program according to the sixth embodiment.

### DETAILED DESCRIPTION

Various embodiments will be described hereinafter with reference to the accompanying drawings.

The disclosure is merely an example and is not limited by contents described in the embodiments described below. Modification which is easily conceivable by a person of ordinary skill in the art comes within the scope of the disclosure as a matter of course. In order to make the description clearer, the sizes, shapes, and the like of the respective parts may be changed and illustrated schematically in the drawings as compared with those in an accurate representation. Constituent elements corresponding to each other in a plurality of drawings are denoted by like reference numerals and their detailed descriptions may be omitted unless necessary.

In general, according to one embodiment, an electronic apparatus includes one or more processors. The one or more processors are configured to acquire first information related to reliability of biological information, calculate reliability information of the biological information based on the first information, and associate the reliability information means with the biological information.

### First Embodiment

[System Configuration]

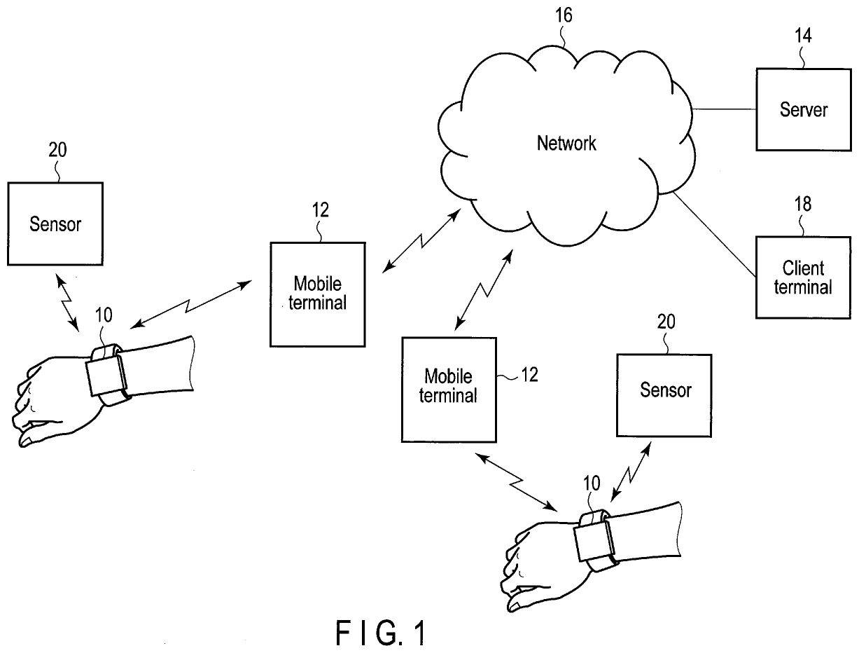

FIG. 1 is a block diagram showing an example of a system including an electronic apparatus according to a first embodiment. The first embodiment relates to a stress determination system. The system calculates a stress index (e.g., LF/HF) for determining the degree of psychological stress from information (e.g., heartbeat interval) indicative of the state of a living body. The system notifies a user or an administrator of the stress index. The system may notify the user or the administrator of the degree of stress that is a result of comparison between the stress index and a threshold.

The first embodiment will be described with reference to psychological stress, and an embodiment relating to physical stress will be described later. In the present specification, stress refers to psychological stress and physical stress, unless otherwise specified in particular.

In the present specification, information indicative of the state of a living body and information that is obtained by calculating the information indicative of the state of the living body will be referred to as biological information.

In the embodiment, the system may notify the user or the administrator of the reliability of the biological information together with the stress index or the degree of stress.

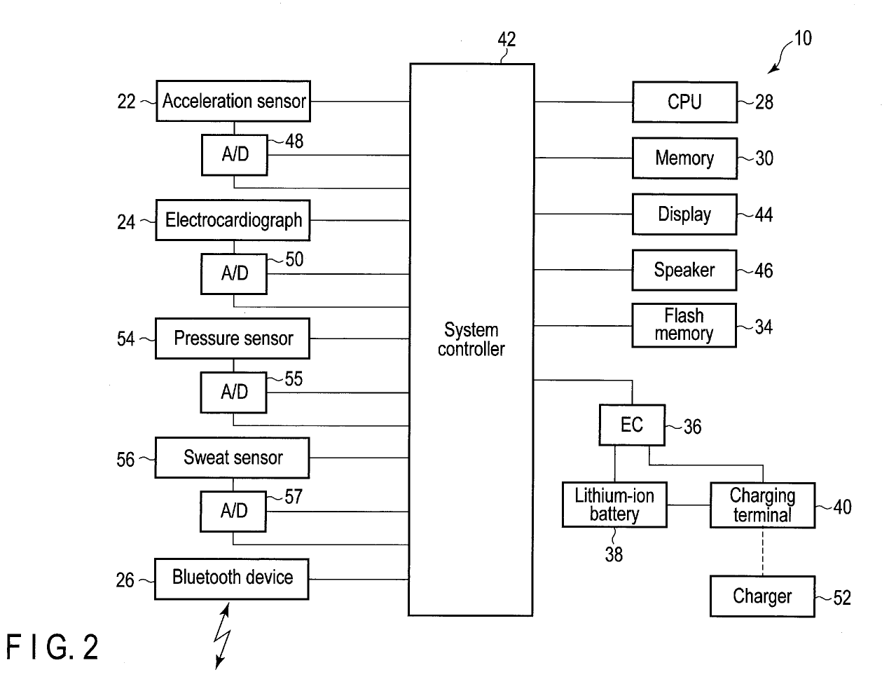

A sensor device 10 that senses various states of a user is attached to the user and is connected to a mobile terminal 12 by a wireless or wired channel. The sensor device 10 is shown in detail in FIG. 2, and may include a plurality of sensors to sense various states of a living body necessary to calculate a stress index and reliability. A sensor 20 may be provided outside the sensor device 10 to sense various states that affect the living body. The sensor 20 may be connected to the sensor device 10 by a wireless or wired channel and may be connected to the mobile terminal 12 by a wireless or wired channel.

One aspect of the sensor device 10 is a wearable device. The aspect of the wearable device may be any of a wristband type, a wristwatch type, a ring type, a pair of glasses type, an earring type, a pendant type, a sticking type, a clothes built-in type, etc., and may be attached to the lower sternum part, etc., by a belt. An aspect of the sensor device 10 other than the wearable device may be a sensor device called a mattress sensor provided on a bed. The mattress sensor is a pressure sensor that is placed on the surface of a bed mattress, measures vibrations of a user's chest or abdomen, and senses user's heartbeat and body motion. The pressure sensor has only to sense the vibrations such that user's absence (a user is waked-up), presence (a user is slept), body motion, etc., can be sensed. The pressure sensor may be composed of a piezoelectric element shaped like a tape by forming a polymer piezoelectric material (e.g., polyvinylidene fluoride) into a thin film and attaching flexible electrode films to both surfaces of the thin film.

The sensor device 10 and the mobile terminal 12 may be connected by a wireless communication such as Bluetooth (registered trademark). The sensor device 10 may include an arithmetic operation unit that processes a sensing result and calculates a stress index and the degree of reliability. In the first embodiment, however, the mobile terminal 12 includes an arithmetic operation unit. The sensor device 10 transmits a sensing result to the mobile terminal 12 and receives a control signal and the like from the mobile terminal 12. The mobile terminal 12 receives sensor data from the sensor device 10 and calculates the stress index and reliability from the received sensor data. The calculation may be performed by the sensor device 10 and in this case, the sensor device 10 transmits the stress index and reliability to the mobile terminal 12.

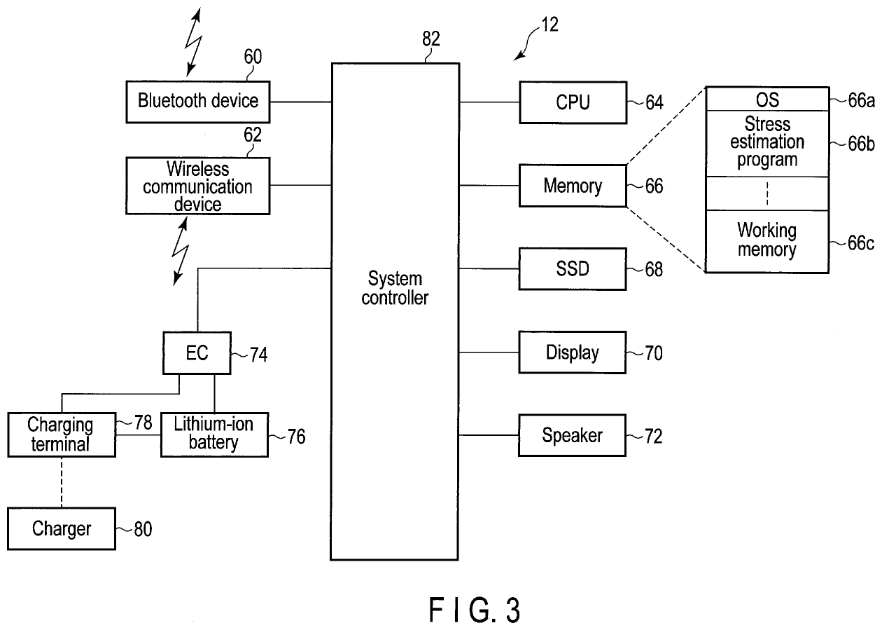

The mobile terminal 12 transmits the stress index and reliability to a server 14 via a network 16. The server 14 determines the degree of stress based on the stress index and reliability and outputs a determination result or transmits the determination result to the mobile terminal 12 via the network 16. The determination result is output from a display 70 (shown in FIG. 3) or a speaker 72 (shown in FIG. 3) of the mobile terminal 12. The degree of stress may be determined by the mobile terminal 12 as well as the server 14. When the sensor device 10 calculates the stress index, the sensor device 10 may determine the degree of stress. Examples of the determination of the degree of stress include “reduction of stress” and “accumulation of stress”. In the case of “accumulation of stress”, the sensor device 10, mobile terminal 12, server 14, or client terminal 18 may give a warning. The mobile terminal 12 may be a mobile phone, a smartphone, a personal computer (PC), a tablet terminal, or the like, or may be a dedicated terminal. Furthermore, various states of a living body necessary to calculate the stress index and reliability and information affecting the states of the living body are not sensed by the sensor device 10 or the sensor 20, but the server 14 may obtain such information by other means and transmit it to the mobile terminal 12 or the sensor device 10. Further, the mobile terminal 12 itself may receive such information.

There may be a plurality of users whose stress is to be checked, and a plurality of sensor devices 10 may be connected to a plurality of mobile terminals 12, respectively. The mobile terminals 12 may be wirelessly connected to the network 16. The server 14 and the client terminal 18 may be connected to the network 16 by a wireless or wired channel. The client terminal 18 may include a personal computer and the like. The server 14 may be connected to a plurality of mobile terminals 12. For example, the server of a company healthcare division receives a stress index from the mobile terminals 12 of the company employees, and stores information useful for determining a stress index and a stress state in their own storages or storages on the network 16. An industrial physician or the like can access to the server 14 using the client terminal 18, browse information regarding the stress of employees and the like, determine the degree of stress, and perform healthcare management according to the degree of stress.

Although FIG. 1 shows a system including a plurality of units of the sensor device 10, sensor 20, mobile terminal 12, client terminal 18, and server 14, the number of units is not limited to the example of FIG. 1. All of the functions of the units shown in FIG. 1 may be implemented in a single unit such as the sensor device 10. The functions of the units shown in FIG. 1 may be divided and implemented in two or three units. Specifically, the functions may be implemented in both the sensor device 10 and the mobile terminal 12 or both the sensor device 10 and the server 14. In FIG. 1, the sensor device 10 transmits electrocardiogram to the mobile terminal 12. The mobile terminal 12 calculates a stress index based on the electrocardiogram and transmits it to the server 14. The server 14 stores the stress index. However, the sensor device 10 may calculate the stress index and store the calculated stress index. Further, the server 14 may be excluded and the mobile terminal 12 may store the stress index, or the mobile terminal 12 may be excluded, and the server 14 may calculate the stress index and store the calculated stress index. The sensor device 10 and the mobile terminal 12 are terminals for each user. Information from a plurality of sensor devices 10 can be uploaded to the server 14 via a plurality of mobile terminals 12.

[Sensor Device 10]

FIG. 2 is a block diagram showing an example of a configuration of the sensor device 10. The sensor device 10 includes various sensors, a Bluetooth device 26, a CPU 28, a memory 30, a flash memory 34, an embedded controller (EC) 36, a secondary battery (for example, a lithium-ion battery) 38, a charging terminal 40, a system controller 42, a display 44, a speaker 46, and the like. Examples of the sensors include an acceleration sensor 22, an electrocardiograph 24, a pressure sensor 54, a sweat sensor 56, and the like. The system controller 42 is a bridge device that connects the CPU 28 and each component.

The electrocardiograph 24 includes an electrode to measure an electrocardiogram, and the sensor device 10 is attached to a user such that the electrode may be in contact with a blood vessel of the user. A heartbeat interval is obtained by extracting a heartbeat (R wave) from the electrocardiogram, and LF/HF is obtained from the heartbeat interval. Instead of the electrocardiograph 24, a pulse wave sensor may be used to measure a pulse and obtain LF/HF from a pulse interval. If a pulse wave sensor is used, the sensor device 10 may include a light emitting element (e.g., a green LED) and a photodiode to measure, as a pulse wave, a change in the volume of a blood vessel that occurs as the heart pumps blood.

The analog electrocardiogram signal output from the electrocardiograph 24 is converted into digital electrocardiogram data by an A/D converter 50 and input to the system controller 42. The A/D converter 50 converts the output current of the electrocardiograph 24 into a voltage, amplifies the voltage, and converts the amplified voltage into electrocardiogram data through a high-pass filter and a low-pass filter. A cutoff frequency of the high-pass filter is, for example, 0.1 Hz and a cutoff frequency of the low-pass filter is, for example, 50 Hz.

The acceleration sensor 22 measures accelerations in three axial directions and senses a body motion of the user based on the total or average of the accelerations in three axial directions. Analog body motion signal is input to the system controller 42 from the acceleration sensor 22 via an A/D converter 48. The A/D converter 48 adjusts the gain and offset of the analog signal and converts the adjusted analog signal into body motion data. The acceleration sensor 22 constantly measures the accelerations, and the A/D converter 48 supplies the body motion data to the system controller 42 at intervals. When the body motion is small, the reliability of measurement results of the electrocardiograph 24 may be determined to be high because the measurement results are hardly affected by noise due to the body motion. When the body motion is large, the measurement results of the electrocardiograph 24 may be determined to be low because they are easily affected by noise due to the body motion.

The pressure sensor 54 senses pressure of contact between the sensor device 10 and the user's skin, and supplies contact pressure data to the system controller 42 at regular intervals via an A/D converter 55. When the contact pressure is moderate, it may be determined that the state of contact between the electrode of the electrocardiograph 24 and the user is satisfactory and the reliability of the measurement results of the electrocardiograph 24 is high. If the contact pressure is too high or too low, it may be determined that the state of contact between the electrode of the electrocardiograph 24 and the user is not satisfactory and the reliability of the measurement results of the electrocardiograph 24 is low.

The sweat sensor 56 senses an amount of sweat of the user and supplies sweat quantity data indicative of the sweat quantity to the system controller 42 via an A/D converter 57 at regular intervals. When the sweat quantity is moderate, the impedance between the electrode of the electrocardiograph 24 and the skin of the user is lowered, and the reliability of measurement results of the electrocardiograph 24 may be determined to be high. When the sweat quantity is too large, the state of contact between the electrode of the electrocardiograph 24 and the skin of the user becomes unsatisfactory. When the sweat quantity is too small, the impedance of the skin surface is increased, and the reliability of the measurement results of the electrocardiograph 24 can be determined to be low. Note that a pulse wave sensor can be used instead of the electrocardiograph 24 as a heartbeat measurement sensor. When the pulse wave sensor is used, if the sweat quantity is too large, the pulse wave sensor cannot measure the sweat quantity correctly because diffused reflection of light occurs, and the reliability of the measurement results is lowered. If the sweat quantity is low or moderate, the reliability can be determined to be high.

The Bluetooth device 26 is used for communications with the mobile terminal 12. The system controller 42 transmits the electrocardiogram data, body motion data, contact pressure data, and sweat quantity data to the mobile terminal 12 using the Bluetooth device 26. The system controller 42 receives a control signal from the mobile terminal 12 using the Bluetooth device 26.

The CPU 28 is a processor that controls the operation of each component of the sensor device 10. The CPU 28 executes an application program (hereinafter referred to simply as a program) stored in the flash memory 34 to control the operation of the sensor device 10. Note that the program can be updated. The operation of the sensor device 10 is not controlled by the program, but may be controlled by dedicated hardware such as a custom LSI, a semi-custom LSI and a programmable DSP.

The embedded controller 36 is a power management controller to manage power of the sensor device 10, and controls charging of the lithium-ion battery 38 as a secondary battery. When the sensor device 10 is mounted on the charger 52, the lithium-ion battery 38 is charged with a charging current flowing from the charger 52 to the sensor device 10 through the charging terminal 40. The embedded controller 36 applies operating power to each component based on the power from the lithium-ion battery 38.

A device ID is given to the sensor device 10. When the sensor device 10 transmits data, such as the electrocardiogram data, body motion data, contact pressure data, and sweat quantity data, to the mobile terminal 12, the device ID is included in the transmit data. The mobile terminal 12 can thus identify data from the sensor device 10 based on the device ID.

[Mobile Terminal 12]

FIG. 3 is a block diagram showing an example of a configuration of the mobile terminal 12. The mobile terminal 12 may include a Bluetooth device 60, a wireless communication device 62, a CPU 64, a memory 66, an SSD (or HDD) 68, a display 70, a speaker 72, an embedded controller (EC) 74, a lithium-ion battery 76 as a secondary battery, a charging terminal 78, a system controller 82, and the like. The system controller 82 is a bridge device that connects the CPU 64 and each component.

The CPU 64 is a processor that controls the operation of each component implemented in the mobile terminal 12. The CPU 64 executes various programs loaded into the memory 66 from the SSD 68 that is a nonvolatile storage device. The programs include an operating system (OS) 66a, a stress estimation program 66b and the like. The memory 66 includes a working memory 66c. The stress estimation program 66b calculates a stress index based on the biological information.

An example of the stress index will be described. As an event related to cardiac motion, there are a heartbeat and a pulse. The number of heartbeats per minute is a heart rate, and the number of beats of arteries in the arms and legs per minute is a pulse rate. In most cases, the heart rate and pulse rate are identical. The following embodiments will be therefore described using the heart rate, but embodiments may be constituted using the pulse rate in place of the heart rate. The inverse of the heart rate (pulse rate) is a heartbeat interval (pulse interval).

The cardiac motion is controlled by the operation of autonomic nerves. The autonomic nerves include sympathetic and parasympathetic nerves. When the sympathetic nerve becomes active (is activated), the heart rate increases. When the parasympathetic nerve becomes active, the heart rate decreases. When a body is moved, the heart rate increases because the sympathetic nerve is activated. When the body is not moving, or when it is at rest, the heart rate decreases because the parasympathetic nerve is activated. The use of the heart rate at rest makes it possible to evaluate autonomic balance with stability. The autonomic balance is affected by stress. Tension activates a sympathetic nerve due to instantaneous stress and increases the heart rate (one's heart beats fast). In addition, when chronic stress due to a job, a human relationship, and the like is generated, the sympathetic nerve is always activated and thus the heart rate at rest increases. Using an autonomic index related to a heartbeat (e.g., a heart rate or a heartbeat interval), a stress index related to the degree of stress can be determined.

The autonomic index is classified into a time domain index and a frequency domain index. The frequency domain index includes LF/HF, HF only, LF only, VLF, total power, etc. The LF is the integral of low-frequency components (e.g., 0.05 Hz to 0.15 Hz) of a power spectrum, which indicates activity of a sympathetic nerve. The HF is the integral of high-frequency components (e.g., 0.15 Hz to 0.40 Hz) of the power spectrum, which indicates activity of a parasympathetic nerve. The LF/HF represents a balance between sympathetic and parasympathetic nerves. When the LF/HF is large, it indicates sympathetic dominance. When the LF/HF is small, it indicates parasympathetic dominance. The autonomic index can thus be used as a stress index. The VLF is the integral of very low-frequency components (about 0 Hz to 0.05 Hz) of the power spectrum. The total power is the total value of power spectra at frequencies of 0 Hz to 0.40 Hz (VLF, LF, and HF) in a short time (e.g., five minutes) test. This value reflects the overall activities of an autonomic nervous system that is dominated by the activity of the sympathetic nerve.

The time domain autonomic index includes HRT, Mean, SDNN, RMSSD, NN50, and pNN 50, and the like. The “HRT” is a mean value of heart rates per minute. The “Mean” is a mean value of heartbeat intervals. The “SDNN” is a standard deviation of heartbeat intervals. The “RMSSD” is the square root of a mean value of the squares of differences between consecutive adjacent heartbeat intervals. The “NN50” is the total number of heartbeat intervals in which a difference between consecutive adjacent heartbeat intervals exceeds 50 milliseconds. The “pNN 50” is the rate of heartbeat in which a difference between consecutive adjacent heartbeat intervals exceeds 50 milliseconds.

The instantaneous stress is calculated using LF/HF measured over a short period (one minute, five minutes, fifteen minutes, etc.). The chronic stress is calculated using LF/HF measured over a long period (one week, one month, or longer).

In the first embodiment, the stress estimation program 66b calculates LF/HF based on electrocardiogram data. Since the electrocardiogram data, the body motion data, contact pressure data, and the sweat quantity data are related to the reliability of LF/HF, the stress estimation program 66b may calculate the reliability of LF/HF based on at least one of the electrocardiogram data, the body motion data, the contact pressure data, and the sweat quantity data. The reliability and the LF/HF may be calculated by any of the sensor device 10, the mobile terminal 12, and the server 14. The reliability and the LF/HF may be displayed on the display 44 of the sensor device 10, the display 70 of the mobile terminal 12, or the client terminal 18 that accesses to the server 14 in association with each other. When the reliability of LF/HF is low, the stress estimation program 66b may determine the cause of the low reliability and present the cause, and/or countermeasures. The cause may be determined by any of the sensor device 10, the mobile terminal 12, and server 14. Information indicative of a result of the determination may also be displayed on the display 44 of the sensor device 10, the display 70 of the mobile terminal 12, or the client terminal 18 that accesses to the server 14 in association with each other. Note that the stress estimation program 66b can be updated.

The calculation of the LF/HF, the calculation of the reliability of LF/HF, and the process to determine the cause of low reliability and present the cause and/or countermeasures may not executed by the stress estimation program 66b, but may be executed by dedicated hardware such as a custom LSI, a semi-custom LSI and a programmable DSP.

The display 70 may display the LF/HF, its reliability, and the cause of low reliability in association with each other, based on a display signal from the system controller 82 under the control of the CPU 64. When the stress state is unsatisfactory or the reliability is low, the display 70 may display a warning. Information and warning about stress may be output from the speaker 72. The display 70 may be a touch panel having an input function. The touch panel may be used to input information indicative of various states of a living body necessary to calculate the LF/HF and its reliability or information indicative of a state that affects the states of the living body to the mobile terminal 12.

The Bluetooth device 60 communicates with the Bluetooth device 26 of the sensor device 10, receives sensor data from the sensor device 10, and transmits a control signal to the sensor device 10.

The wireless communication device 62 is a module configured to perform wireless communication, such as wireless LAN and 3G/4G mobile communication, or near field wireless communication, such as near field communication (NFC). The mobile terminal 12 is connected to the network 16 via the wireless communication device 62. When the server 14 or the client terminal 18 includes an input device, items of input information may be transmitted to the mobile terminal 12 via the wireless communication device 62. The input device is configured to input information indicative of various states of a living body required to calculate the LF/HF and its reliability or information indicative of a state that affects the states of a living body.

The embedded controller 74 is a power management controller to manage power of the mobile terminal 12 in the same manner as the embedded controller 36 of the sensor device 10, and controls charging of the lithium-ion battery 76. When the mobile terminal 12 is attached to the charger 80, a charging current flows from the charger 80 to the mobile terminal 12 through the charging terminal 78 to charge the lithium-ion battery 76. The embedded controller 74 applies operating power to each component based on the power from the lithium-ion battery 76.

[Stress Estimation Program]

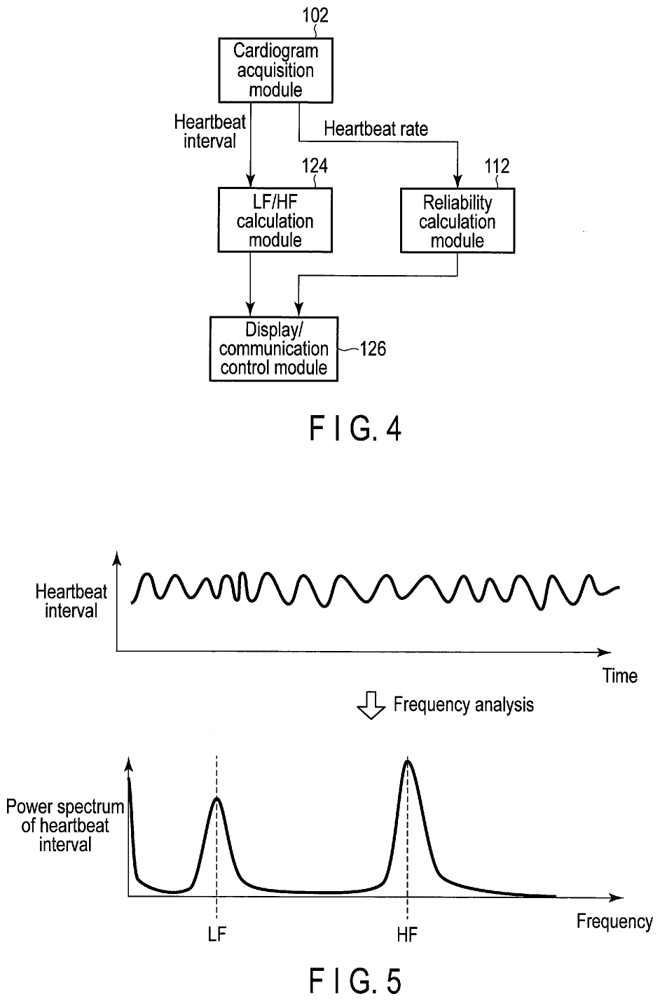

FIG. 4 shows an example of a functional block of the stress estimation program 66b according to the first embodiment. The stress estimation program 66b shown in FIG. 4 includes a cardiogram acquisition module 102, an LF/HF calculation module 124, a reliability calculation module 112, and a display/communication control module 126. Electrocardiogram data output from the electrocardiograph 24 is input to the cardiogram acquisition module 102 via the A/D converter 50. The cardiogram acquisition module 102 detects an R wave from the electrocardiogram data, extracts a heartbeat interval from the period of the R wave, and supplies heartbeat interval data to the LF/HF calculation module 124. The LF/HF calculation module 124 calculates LF and HF using the heartbeat interval data, and supplies LF/HF data to the display/communication control module 126.

As shown in FIG. 5, the LF/HF calculation module 124 converts the heartbeat interval data into frequency/power spectrum distribution by a frequency analysis method such as Fast Fourier Transform (FFT). The LF/HF calculation module 124 integrates low and high frequency components of the power spectrum to calculate autonomic indices LF and HF as stress indices. Using the FFT as a frequency analysis method has an advantage of reducing the burden of data processing. However, any other method, such as the AR model method, the maximum entropy method, the wavelet method, and the MEM method, can be used. In the first embodiment, the power ratio LF/HF of the autonomic indices LF and HF is used as a stress index by using the fact that the influence of HF and LF waves fluctuating with a change of the pulse rate varies depending on the balance of the activity states of the sympathetic and parasympathetic nerves.

The cardiogram acquisition module 102 also detects a heart rate from the electrocardiogram data and supplies heart rate data to the reliability calculation module 112. The reliability calculation module 112 calculates reliability using the heart rate data and supplies reliability data to the display/communication control module 126. When the reliability is low, its value is set small, and when the reliability is high, its value is set large. If the detection of an R wave using the electrocardiograph 24 fails due to, for example, poor contact between the electrode of the electrocardiograph 24 and the user's skin, the heartbeat interval is extended and the heart rate is lowered. If, therefore, the heart rate for a predetermined period (e.g., for one minute) is equal to or lower than a predetermined rate, the heart rate may not be correctly detected, and the reliability of the electrocardiogram data is considered to be low. Thus, the reliability calculation module 112 compares the heart rate for the predetermined period with the predetermined rate, and calculates low reliability when the heart rate is equal to or lower than the predetermined rate. The reliability calculation module 112 supplies the reliability data to the display/communication control module 126. Note that the reliability calculation module 112 may calculate the reliability based on the heartbeat interval as well as the heart rate.

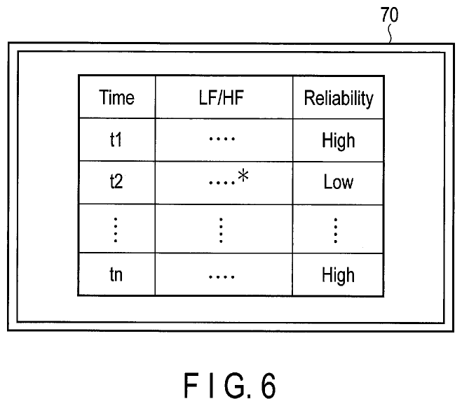

The display/communication control module 126 causes the display 70 to display the LF/HF and the reliability in association with each other and supplies them to the server 14 via the wireless communication device 62. The LF/HF and the reliability supplied to the server 14 may be stored in the server 14 and displayed on the client terminal 18. FIG. 6 shows a display example of the display 70. The reliability and the LF/HF are displayed every time the LF/HF is calculated. Since the reliability is calculated in two stages of high and low, it is displayed in two stages of high and low in the example of FIG. 6. However, when a plurality of thresholds are used, the reliability may be displayed in three or more stages. When the reliability that continuously changes from 0% to 100% (0% represents the lowest reliability and 100% represent the highest reliability) is calculated, its value may be displayed. The LF/HF calculated based on the electrocardiogram data obtained when the reliability is low, may be displayed distinguishably from others, for example, with an asterisk in the upper right, by being surrounded by a frame, in a different color, and by being blinked. In this case, even if the reliability itself is not displayed, it can be recognized that the reliability is low only by displaying the LF/HF. The display example of the client terminal 18 may be the same as that of the display 70.

The electrocardiograph 24 outputs electrocardiogram data and the cardiogram acquisition module 102 acquires electrocardiogram data to extract the heartbeat interval and the heart rate from the acquired electrocardiogram data. Instead, the electrocardiograph 24 may extract the heartbeat interval and the heart rate, and a heartbeat interval/heart rate acquisition module may be provided in place of the cardiogram acquisition module 102.

The example displayed by the display/communication control module 126 is not limited to the example of FIG. 6. The display example may include a continuous display of at least a degree of reliability calculated by the reliability calculation module 112, a degree of reliability in a plurality of stages corresponding to the degree of reliability calculated by the reliability calculation module 112, or words (text, number, etc.) and expressions (mark, icon, etc.) that correspond to the meaning of the reliability.

The display/communication control module 126 may display the biological information (LF/HF) calculated by the LF/HF calculation module 124 in addition to the reliability. Like the reliability, the biological information may continuously be displayed as at least a value thereof, a level thereof in a plurality of stages corresponding to the value thereof, or words (text, number, etc.) and expressions (mark, icon, etc.) that correspond to the meaning of the biological information. In FIG. 6, the LF/HF and the reliability at a plurality of times are displayed, however, only the LF/HF and the reliability at the latest time or a certain time in the past may be displayed.

As described above, the stress estimation program 66b can calculate the LF/HF, which is a stress index, using the heartbeat interval data extracted from the electrocardiogram data acquired by the cardiogram acquisition module 102. The stress estimation program 66b can further calculate the reliability of the electrocardiogram data, namely, the reliability of LF/HF, using the heart rate data extracted from the electrocardiogram data. In the conventional system, the heartbeat interval data acquired when it is determined that “the body is moving” is canceled without being used for the calculation of LF/HF. Thus, the heartbeat interval data is lost and the calculation of the LF/HF is interrupted. However, even if the body is moving, the heartbeat interval data may correctly be acquired. In the first embodiment, therefore, the acquired heartbeat interval data is not canceled. The LF/HF calculation module 124 calculates the LF/HF based on all items of heartbeat interval data extracted from the acquired electrocardiogram data. The LF/HF calculation module 124 can thus calculate the LF/HF without interruption. The display/communication control module 126 can display the LF/HF and its reliability together. The user can determine the degree of stress using all items of the calculated LF/HF including the LF/HF calculated when the body is moved, while considering their reliabilities. The LF/HF with low reliability can be used as a reference value to determine the degree of stress.

### Second Embodiment

In the first embodiment, the reliability is calculated by the heart rate (a single item of information). In the second embodiment, a plurality of reliabilities are calculated by a plurality of items of information related to the reliability of LF/HF. The system configuration, the sensor device 10, and the mobile terminal 12 of the second embodiment are the same as those of the first embodiment shown in FIGS. 1, 2 and 3. The second embodiment differs from the first embodiment in the stress estimation program 66b executed by the mobile terminal.

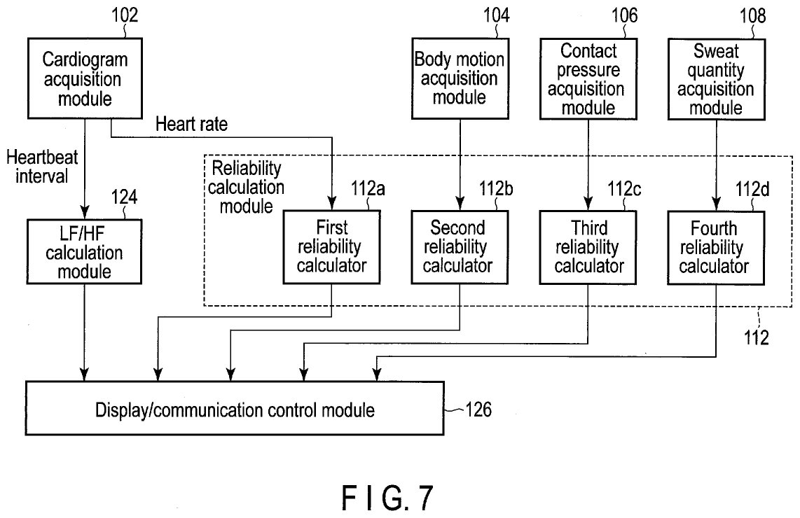

FIG. 7 shows an example of a functional block of the stress estimation program 66b executed by the mobile terminal 12 of the second embodiment. The stress estimation program 66b shown in FIG. 7 includes a cardiogram acquisition module 102, a body motion acquisition module 104, a contact pressure acquisition module 106, a sweat quantity acquisition module 108, an LF/HF calculation module 124, a reliability calculation module 112, and a display/communication control module 126. The reliability calculation module 112 includes a first reliability calculator 112a, a second reliability calculator 112b, a third reliability calculator 112c, and a fourth reliability calculator 112d.

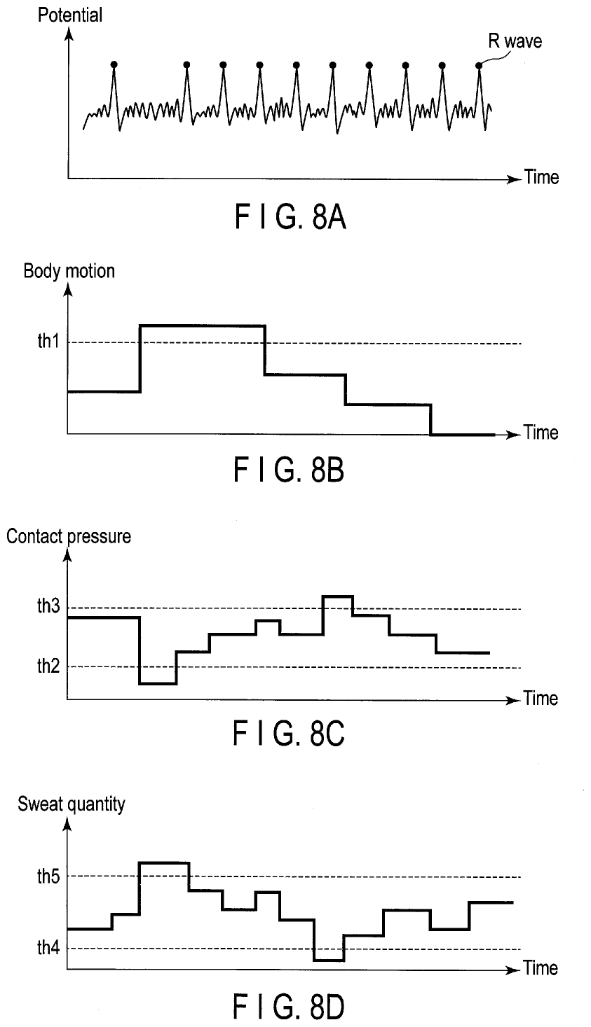

The cardiogram acquisition module 102 detects an R wave from electrocardiogram data as shown in FIG. 8A, extracts a heartbeat interval from the period of the R wave, supplies heartbeat interval data to the LF/HF calculation module 124, detects a heart rate from the electrocardiogram data, and supplies heart rate data to the first reliability calculator 112a. FIG. 8B shows the body motion data with regard to the electrocardiogram (FIG. 8A). FIG. 8C shows the contact the pressure data with regard to the electrocardiogram (FIG. 8A). FIG. 8D shows the sweat quantity data with regard to the electrocardiogram (FIG. 8A).

Like in the first embodiment, the first reliability calculator 112a calculates first reliability based on the number of items of the heart rate data as shown in FIG. 8A and supplies first reliability data to the display/communication control module 126.

The body motion data is input to the body motion acquisition module 104 from the acceleration sensor 22. The body motion acquisition module 104 supplies the body motion data to the second reliability calculator 112b. When the body is moved, the reliability of the calculated LF/HF is considered low because it is influenced by noise due to the body motion. As shown in FIG. 8B, therefore, the second reliability calculator 112b compares the body motion with a threshold th1. When the body motion is equal to or larger than the threshold th1, the second reliability calculator 112b calculates a low reliability. Thus, the second reliability calculator 112b calculates second reliability based on the body motion data, and supplies second reliability data to the display/communication control module 126.

The contact pressure data is input to the contact pressure acquisition module 106 from the pressure sensor 54. The contact pressure acquisition module 106 supplies the acquired contact pressure data to a third reliability calculator 112c. Even if the contact pressure is too low or too high, the reliability of the calculated LF/HF is considered low because the electrode of the electrocardiograph 24 is not properly in contact with the user's skin and thus an electrocardiogram data cannot accurately be obtained. As shown in FIG. 8C, therefore, the third reliability calculator 112c compares the contact pressure with a lower threshold th2 and an upper threshold th3. When the contact pressure is equal to or lower than the lower threshold th2 or when it is equal to or higher than the upper threshold th3, the third reliability calculator 112c calculates a low reliability. Thus, the third reliability calculator 112c calculates third reliability based on the comparison between the contact pressure and the thresholds, and supplies third reliability data to the display/communication control module 126.

The sweat quantity data is input to the sweat quantity acquisition module 108 from the sweat sensor 56. The sweat quantity acquisition module 108 supplies the sweat quantity data to the fourth reliability calculator 112d. Even if a sweat quantity is too small or too large, the reliability of the calculated LF/HF is considered low because an electrocardiogram data cannot accurately be obtained when the electrocardiograph 24 is used. As shown in FIG. 8D, therefore, the fourth reliability calculator 112d compares a sweat quantity with a lower threshold th4 and an upper threshold th5. When the sweat quantity is equal to or smaller than the lower threshold th4 or when the sweat quantity is equal to or larger than the upper threshold th5, the fourth reliability calculator 112d calculates a low reliability. Thus, the fourth reliability calculator 112d calculates fourth reliability based on the comparison between the sweat quantity data and the thresholds, and supplies fourth reliability data to the display/communication control module 126.

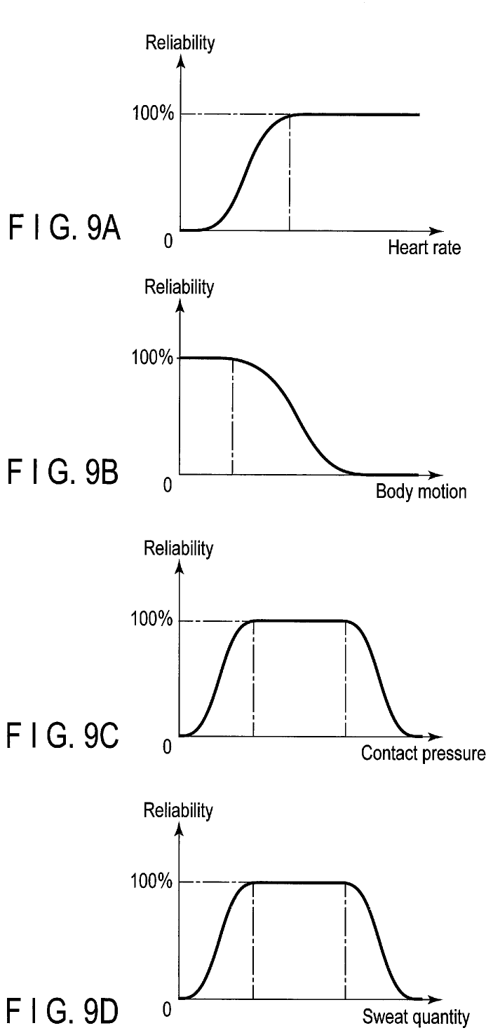

In the above-described reliability calculation method, the information regarding reliability is compared with a threshold to calculate two reliabilities (low and high reliabilities). A method of calculating reliability that varies continuously will be described. As shown in FIG. 9A, there is following relationship between the reliability and the heart rate. When the heart rate is 0, the reliability is 0%, the reliability increases as the heart rate increases, and the reliability is 100% when the heart rate is equal to or higher than a threshold. As shown in FIG. 9A, the first reliability calculator 112a can set a function in which the reliability increases as the heart rate increases and converges to a value (e.g., 100%) determined to be the maximum, and can calculate the first reliability using this function.

Similarly, as shown in FIG. 9B, there is following relationship between the reliability and an amount of body motion. When the amount of body motion is 0, the reliability is 100%, and the reliability decreases and converges to 0% as the amount of body motion increases. The second reliability calculator 112b can calculate second reliability based on the inverse of the amount of the body motion or the proportion of the amount of the body motion in the LF/HF calculation section, which is equal to or lower than a threshold. In addition, as shown in FIG. 9B, the second reliability calculator 112b can set a function in which the reliability increases as the amount of the body motion decreases and converges to a value (e.g., 100%) determined to be the maximum, and can calculate the second reliability using this function.

Furthermore, as shown in FIG. 9C, there is following relationship between the reliability and the contact pressure. The reliability is low even if the contact pressure is too high or too low, and the reliability is the highest when the contact pressure is moderate. As shown in FIG. 9C, the third reliability calculator 112c can set a function in which the reliability is a value (e.g., 0%) determined to be the minimum when the contact pressure is 0, the reliability increases as the contact pressure increases, and the reliability has a value (e.g., 100%) determined to be the maximum when the contact pressure is in the range from a first threshold to a second threshold, and the reliability gradually decreases and converges to a value determined to be the minimum when the contact pressure exceeds the second pressure. The third reliability calculator 112c can set third reliability using this function.

Furthermore, as shown in FIG. 9D, there is the following relationship between the reliability and the sweat quantity. The reliability is low even if the sweat quantity is too small or too large and it is the highest when the sweat quantity is moderate. As shown in FIG. 9D, the fourth reliability calculator 112d sets a function in which the reliability is a value (e.g., 0%) determined to be the minimum when the sweat quantity is 0, the reliability increases as the sweat quantity increases, the reliability is a value (e.g., 100%) determined to be the maximum when the sweat quantity is in the range from a first threshold to a second threshold, and the reliability gradually decreases and converges to a value determined to be the minimum when the sweat quantity exceeds the second threshold. The fourth reliability calculator 112d can calculate fourth reliability using this function.

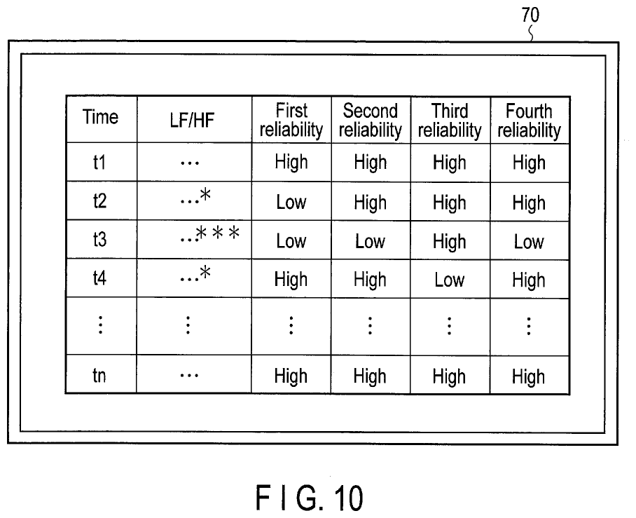

Similarly to the first embodiment, the display/communication control module 126 may cause the display 70 to display the first to fourth reliabilities, which are calculated by the first to fourth reliability calculators 112a to 112d, in association with the LF/HF, and supply them to the server 14 via the wireless communication device 62. FIG. 10 shows a display example of the display 70. The first to fourth reliabilities and the value of the LF/HF are displayed every time the LF/HF is calculated. In the example of FIG. 10, the reliability is displayed in two stages of high and low, but it may be displayed in three or more stages when a plurality of thresholds are used. When the reliabilities that continuously change from 0% to 100% as shown in FIGS. 9A to 9D are calculated, its values may be displayed. The LF/HF calculated based on the electrocardiogram data obtained when the reliability is low, may be displayed distinguishably from others. For example, the LH/HF may be displayed with an asterisk in the upper right, by being surrounded by a frame, in a different color, and by being blinked. When the LF/HF is displayed with asterisks, the number of asterisks may correspond to the number of items of the information related to the calculated low reliability. In this case, even if the reliability itself is not displayed, it can be recognized that the reliability is low only by displaying the LF/HF.

According to the second embodiment, the reliability of LF/HF or the reliability of the heart rate used for the calculation of LF/HF that is a stress index can also be displayed together with the LF/HF. Therefore, the LF/HF can be calculated without interruption, and the degree of stress can be determined using all items of the calculated LF/HF in consideration of the reliability of LF/HF.

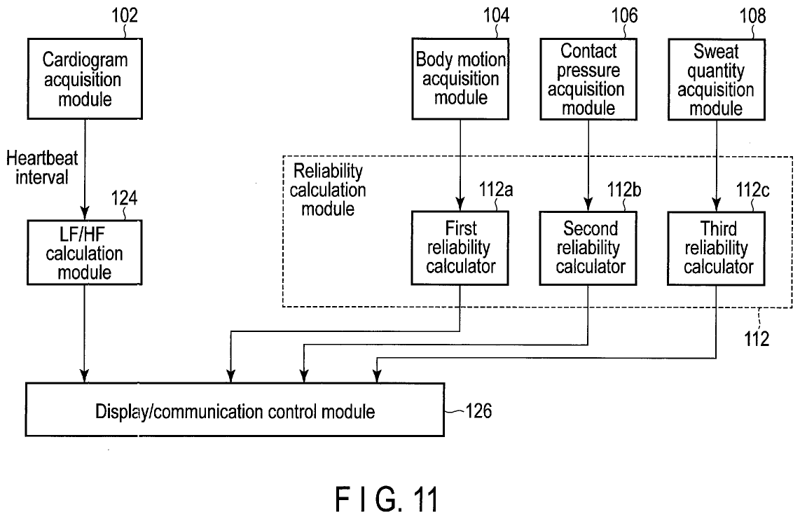

In the second embodiment, the heart rate is used as the information related to reliability. As shown in FIG. 11, however, the reliability may be calculated based on a plurality of items of information (e.g., the body motion data, the contact pressure data, and the sweat quantity data) other than the heart rate. In this case, the reliability calculation module 112 includes the first, the second, and the third reliability calculators 112a, 112b, and 112c. Data items acquired by the body motion acquisition module 104, the contact pressure acquisition module 106, and the sweat quantity acquisition module 108 are input to the first, second, and third reliability calculators 112a, 112b and 112c.

### Third Embodiment

In the second embodiment, a plurality of reliabilities are calculated and displayed in association with the stress index, but the cause of lowering the reliability of the stress index and/or the countermeasures to eliminate the cause is not determined. A third embodiment in which a user is informed of the cause and/or the countermeasures will be described. The configurations of the system, the sensor device 10, and the mobile terminal 12 of the third embodiment are the same as those of the first embodiment shown in FIGS. 1, 2 and 3. The third embodiment differs from the first and second embodiments in the stress estimation program 66b executed by the mobile terminal 12.

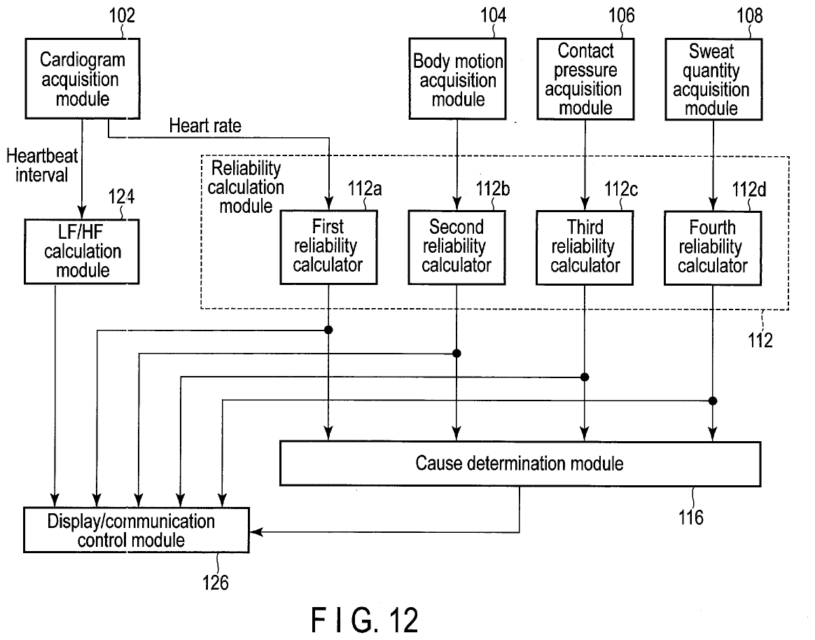

FIG. 12 shows an example of a functional block of the stress estimation program 66b executed by the mobile terminal 12 of the third embodiment. In the stress estimation program 66b shown in FIG. 12, a cause determination module 116 is added to the functional block of the second embodiment shown in FIG. 7. The cause determination module 116 is supplied with the outputs of the first, second, third, and fourth reliability calculators 112a, 112b, 112c, and 112d. The cause determination module 116 determines a cause to lower the reliability of the stress index, and supplies a result of the determination to the display/communication control module 126.

In the cause determination, a cause of low reliability is determined to be a cause to lower the reliability, and the contents of the cause and its countermeasures are output. The method of selecting a low reliability includes a method of selecting reliability whose value is lower than a threshold, a method of selecting the lowest reliability, a method of selecting some lower reliabilities, and the like.

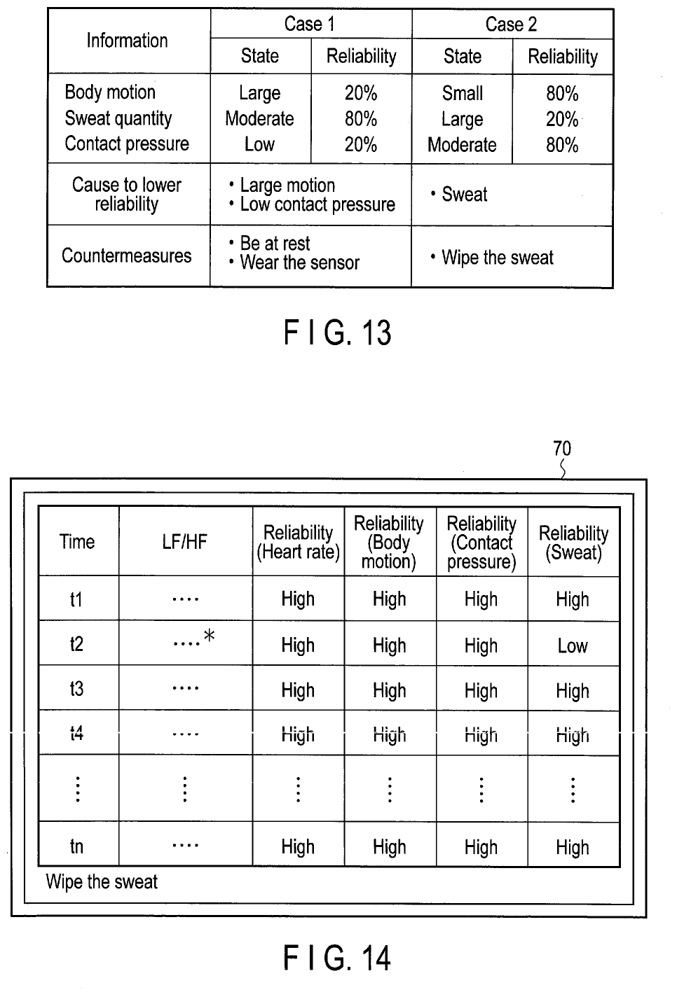

An example of the cause determination process of the cause determination module 116 will be described with reference to FIG. 13. FIG. 13 shows reliabilities corresponding to the body motion, the sweat quantity, and the contact pressure.

In case 1 where the body motion is large, the sweat quantity is moderate and the contact pressure is low, the reliability calculated from the body motion is 20%, the reliability calculated from the sweat quantity is 80%, and the reliability calculated from the contact pressure is 20%. The cause determination module 116 determines from these reliability values that the cause to lower the reliability of the stress index is a large movement and a low contact pressure. The user is thus notified that the user should be at rest and that the sensor (electrode) should be brought into close contact with the user, as countermeasures. An example of the notification may displaying of text and outputting of synthetic speech.

In case 2 where the body motion is small, the sweat quantity is large, and the contact pressure is moderate, the reliability calculated from the body motion is 80%, the reliability calculated from the sweat quantity is 20%, and the reliability calculated from the contact pressure is 80%. The cause determination module 116 determines from these reliability values that the cause to lower the reliability of the stress index is sweat. The user is thus notified that the user should wipe the sweat as countermeasures.

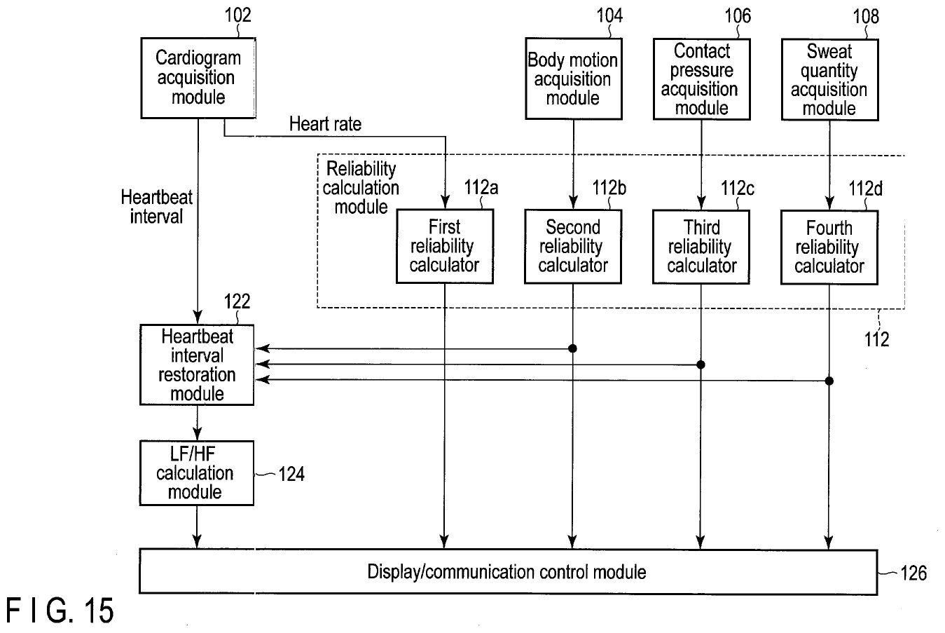

FIG. 14 shows a display example of the display 70. The example where the value of LF/HF and the first to fourth reliabilities are displayed every time the LF/HF is calculated, is the same as the example of FIG. 10. In the third embodiment, countermeasures to eliminate the cause to lower the reliability, such as “wiping the sweat” are also displayed. Note that the countermeasures are not limited to one action and if there are some causes, their corresponding countermeasures are displayed. If there is no low reliability, it is possible to output a message to the effect that the reliability is sufficient or display nothing. In addition to or in place of the display, synthetic speech representing the countermeasures may be output.

According to the third embodiment, in addition to the operation of the second embodiment, a cause to lower the reliability is determined from a plurality of reliabilities, and a result of the determination is displayed, with the result that the user can eliminate the cause to lower the reliability.

### Fourth Embodiment

In the foregoing embodiments, the stress index is calculated using the heartbeat interval extracted from the electrocardiogram, but the heartbeat interval may be calculated to restore a missing heart rate (R wave). The fourth embodiment can be combined with one or more of the first to third embodiments. As one example of the combination, the fourth embodiment will be described in which a heartbeat interval restoration function is added to the second embodiment.

FIG. 15 shows an example of a functional block of the stress estimation program 66b to be executed by the mobile terminal 12 according to the fourth embodiment. The stress estimation program 66b shown in FIG. 15 is obtained by adding a heartbeat interval restoration module 122 to the functional block of the second embodiment shown in FIG. 7. Heartbeat interval data acquired by the cardiogram acquisition module 102 is input to the heartbeat interval restoration module 122. A missing heart rate is restored by the heartbeat interval restoration module 122, and the heartbeat interval data whose heartbeat interval has been restored is input to the LF/HF calculation module 124. The second to fourth reliability data calculated by the second to fourth reliability calculators 112b, 112c, and 112d are also input to the heartbeat interval restoration module 122. The restoration operation of the heartbeat interval restoration module 122 is controlled in accordance with the second to fourth reliability data. When the reliability is low, the restoration operation is performed. When the reliability is high, the restoration operation is not performed.

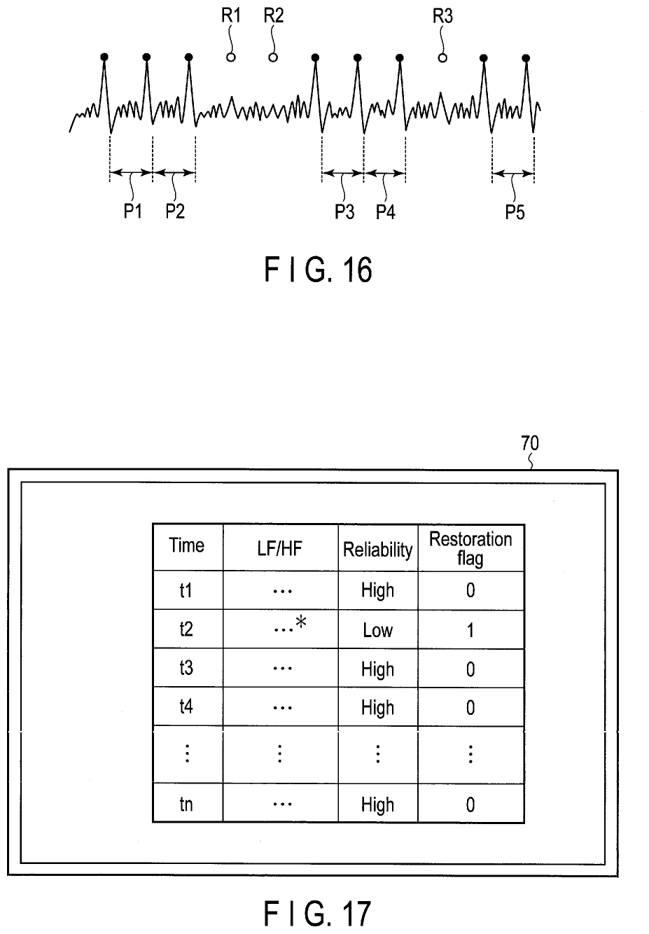

FIG. 16 shows an example of the operation of the heartbeat interval restoration module 122. A heart rate repeats at approximately regular intervals P1, P2, P3, P4, P5, . . . . The heartbeat interval may be longer if the electrocardiograph 24 does not correctly detect an R wave. For example, when neither of two heart rates R1 and R2 is detected, the heartbeat interval is three times as long as the average of the other intervals. When one heart rate R3 is not detected, the heartbeat interval is two times as long as the average of the other intervals. Therefore, when the electrocardiograph 24 does not detect the R wave and the heartbeat interval is longer than the average of the other intervals, the heartbeat interval restoration module 122 can restore the undetected R wave using the average of the other intervals to make it possible to restore the heartbeat interval data.

In the foregoing case, it is assumed that the electrocardiograph 24 does not detect R waves. The heartbeat interval of a living body may vary. That is, the overall heartbeat interval may be increased two times, three times, etc., and decreased to one half, one third, etc. In this case, it may be better not to restore heartbeat interval data. The example shown in FIG. 16 includes a case where the electrode of the electrocardiograph 24 is displaced from a blood vessel and the contact between the electrode and the skin becomes poor. In this case, the electrocardiograph 24 malfunctions. The reliability based on the malfunction of the electrocardiograph 24 is low. Thus, the heartbeat interval restoration module 122 performs a heartbeat interval data restoration operation when the second to fourth reliabilities calculated by the second to fourth reliability calculators 112b, 112c, and 112d are low. The heartbeat interval restoration module 122 does not perform the restoration operation when the second to fourth reliabilities are high. The heartbeat interval restoration module 122 does not restore the heartbeat interval when the heartbeat interval varies as a whole, but restores heartbeat interval data only when the electrocardiograph 24 malfunction, that is, only when the reliability is lowered due to a large body motion, a low contact pressure, or a large sweat quantity.

FIG. 17 shows a display example of the display 70. The value of LF/HF, reliability and a restoration flag are displayed every time the LF/HF is calculated. As the reliability, one reliability is displayed for convenience of illustration, but first to fourth reliabilities may be displayed as in FIG. 14. The restoration flag indicates whether the heartbeat interval data used to calculate the LF/HF is heartbeat interval data restored by the heartbeat interval restoration module 122. It is thus possible to determine whether the calculated LF/HF should be used as information for stress estimation based on the reliability and the restoration flag.

### Fifth Embodiment

In the second to fourth embodiments, a plurality of reliabilities are calculated based on a plurality of items of information related to the reliability of a stress index, and the reliabilities are displayed independently. A fifth embodiment in which the reliabilities are integrated into one reliability will be described. The fifth embodiment can be combined with one or more of the second to fourth embodiments. As an example, the fifth embodiment is combined with the second and third embodiments and has a reliability integration function.

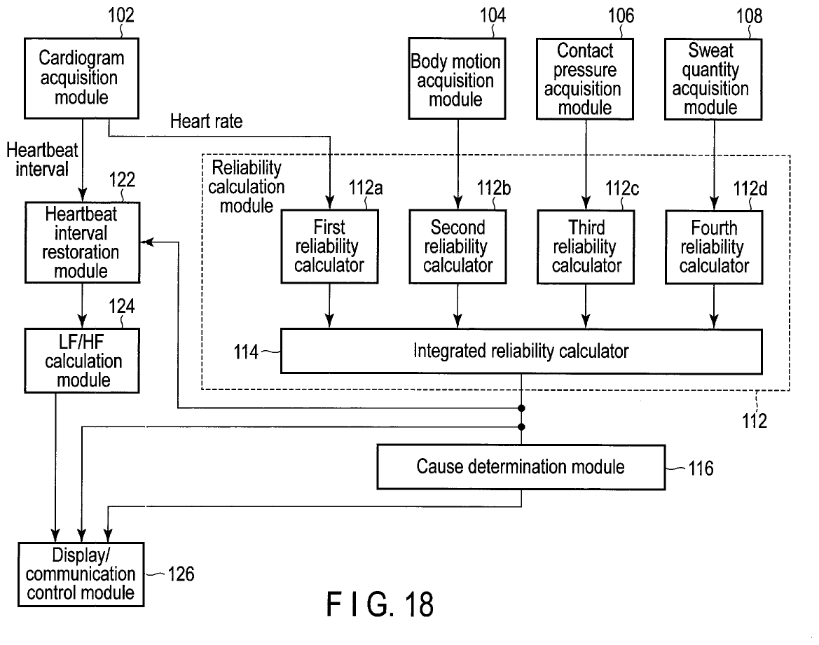

FIG. 18 shows an example of a functional block of a stress estimation program to be executed by the mobile terminal of the fifth embodiment. The reliability calculation module 112 of the stress estimation program shown in FIG. 18 includes the first to fourth reliability calculators 112a to 112d and an integrated reliability calculator 114. The integrated reliability calculator 114 calculates integrated reliability based on the first to fourth reliabilities output from the first to fourth reliability calculators 112a to 112d, and supplies integrated reliability data to the cause determination module 116, the heartbeat interval restoration module 122, and the display/communication control module 126.

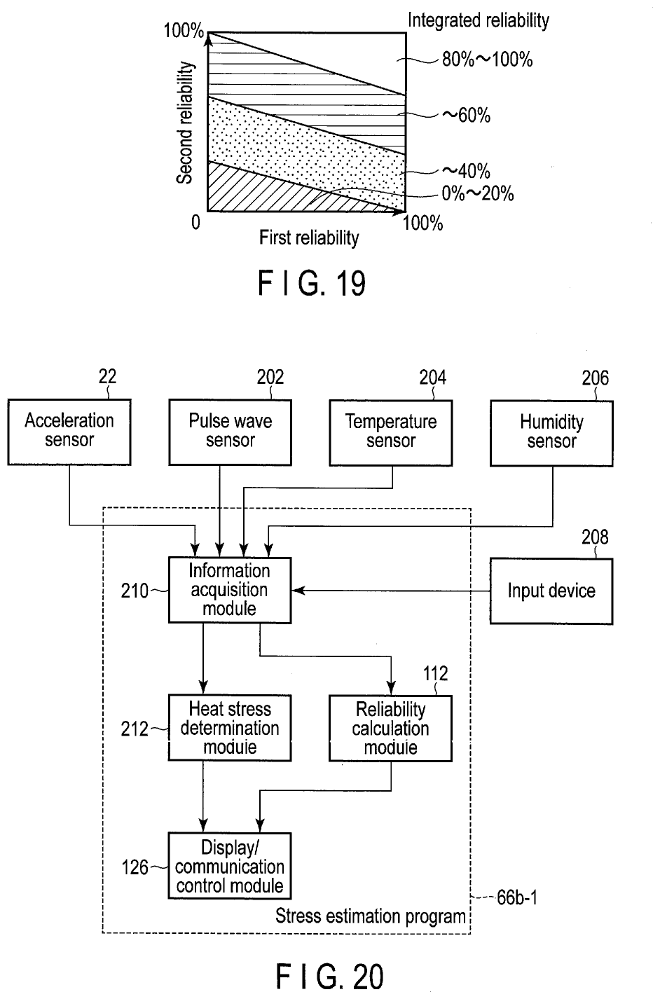

The integrated reliability calculator 114 calculates one integrated reliability based on a plurality of reliabilities. The integrated reliability calculator 114 calculates an integrated reliability by, for example, weighting two reliabilities. FIG. 19 shows an example in which the integrated reliability calculator 114 calculates integrated reliability z (=ax by, where a=0.2, b=0.8) by weighting first reliability x and second reliability y.

In weighting, the heart rate has a greater influence on LF/HF accuracy than the body motion and thus the integrated reliability calculator 114 may set a great weight to the heart rate. Even though the first reliability based on the body motion is low, the integrated reliability is considered high if the second reliability based on the heart rate is high. Examples of integration other than the weighting may include arithmetic mean, trimmed mean (calculation of the mean excluding data near the minimum and maximum values), and the like for two reliabilities. In addition, binary determination as to whether reliability is present or absent may be made using a threshold for each reliability. A ratio of presences of reliability to the all reliabilities may be used as integrated reliability.

When the integrated reliability calculator 114 is supplied with three or more reliabilities, it can calculate the integrated reliability as in the case of two input reliabilities. That is, weighting, arithmetic mean, trimmed average (calculation of the mean excluding data near the minimum and maximum values), and the like may be used for three or more reliabilities. In addition, binary determination as to whether reliability is present or absent may be made using a threshold for each reliability. A ratio of presences of reliability to the all reliabilities may be used as integrated reliability.

### Sixth Embodiment

The first to fifth embodiments relate to the reliability of psychological stress determination. Like the psychological stress determination, physical stress determination is influenced by noise. The sixth embodiment, which relates to the reliability of physical stress determination, will be described. It is assumed in the sixth embodiment that an example of physical stress is heat stress and an example of the index of the heat stress is WBGT.

FIG. 20 shows an example of a functional block of a stress estimation program 66b-1 of the sixth embodiment. The stress estimation program 66b-1 includes an information acquisition module 210, a heat stress determination module 212, the reliability calculation module 112, and the display/communication control module 126.

The information acquisition module 210 is provided in place of the cardiogram acquisition modules 102 of the first to fifth embodiments. The heat stress determination module 212 is provided in place of the LF/HF calculation module 124 of the first to fifth embodiments. The reliability calculation module 112 and the display/communication control module 126 are the same as those of the first to fifth embodiments.

The information acquisition module 210 acquires a body motion (times) from the acceleration sensor 22, a pulse rate (bpm) from a pulse wave sensor 202, temperature (degree) from a temperature sensor 204, and humidity (%) from a humidity sensor 206 periodically, for example, every one minute. The pulse wave sensor 202 may be provided in the sensor device 10 as in the electrocardiograph 24 of the first to fifth embodiments, or may be provided as an independent device. The temperature sensor 204 and the humidity sensor 206 may also be provided in the sensor device 10, or may be provided as independent devices. The temperature sensor 204 measures natural dry bulb temperature, natural wet bulb temperature, and globe temperature.

The sensor device 10 or the mobile terminal 12 is provided with an input device 208, through which age “A” and risk factor “R” of a subject are input to the information acquisition module 210. The risk factor “R” is a heat stroke risk factor of the subject and includes three items. The three items are a past medical history of illness with a high risk of heatstroke, a high BMI, and no exercise habits. The risk factor “R” may be obtained in advance from the subject through questionnaires and the like.

The information acquisition module 210 supplies a pulse rate to the reliability calculation module 112. Since the pulse rate is equal to the heart rate, the reliability calculation module 112 calculates the reliability of biological information based on the pulse rate in place of the heart rate, as in the first to fifth embodiments.

The information acquisition module 210 supplies the heat stress determination module 212 with temperature data, humidity data, the body motion data, a risk factor “R”, a pulse rate, and age “A”. The result of determination of the heat stress determination module 212 is supplied to the display/communication control module 126. The output of the reliability calculation module 112 is supplied to the display/communication control module 126, as in the first to fifth embodiments.

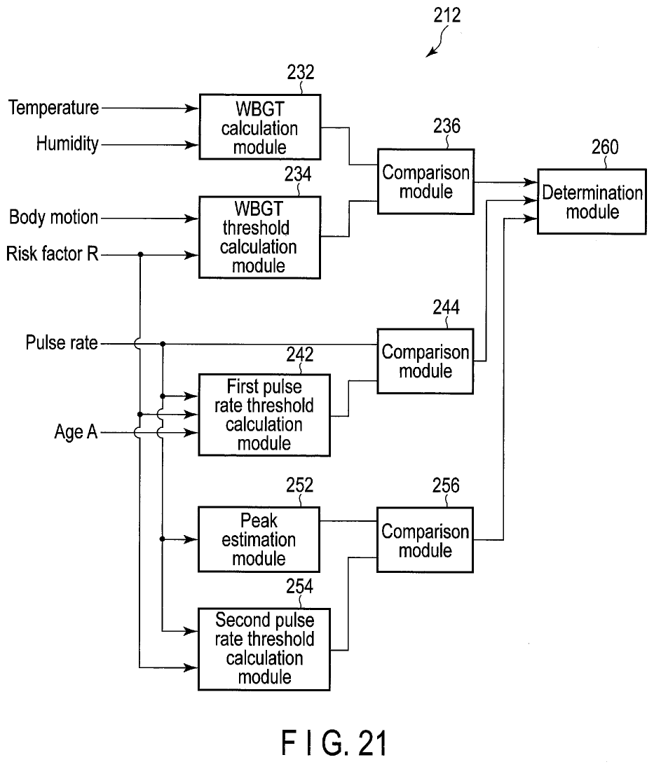

FIG. 21 shows an example of the heat stress determination module 212.

The temperature and humidity are input to a WBGT calculation module 232. The WBGT calculation module 232 calculates the following WBGT value periodically, for example, every one minute.

(Indoors or Outdoors without Solar Radiation)

WBGT value=0.7×natural wet bulb temperature+0.3×globe temperature.

(Outdoors with Solar Radiation)

WBGT value=0.7×natural wet bulb temperature+0.2×globe temperature+0.1×natural dry bulb temperature.

The body motion data and the risk factor “R” are input to a WBGT threshold calculation module 234. The WBGT threshold calculation module 234 calculates a WBGT threshold as follows.

WBGT threshold=WBGT reference value−α1R+β1

where the WBGT reference value is a WBGT value determined by the Ministry of Health, Labor, and

Welfare in Japan according to an exercise category, the exercise category is estimated from the body motion.

β1 is a fixed value determined according to a hot environment.

The WBGT value and the WBGT threshold are compared by a module 236. A result of the comparison is input to a determination module 260.

The pulse rate, risk factor “R”, and age “A” are input to a first pulse rate threshold calculation module 242. The first pulse rate threshold calculation module 242 calculates a first pulse rate threshold as follows.

First pulse rate threshold=(180−A)−α2R+β2

where β2 is a fixed value determined according to a hot environment.

A comparison module 244 compares the pulse rate and the first pulse rate threshold. A result of the comparison is input to the determination module 260.

The pulse rate is input to a peak estimation module 252. The peak estimation module 252 determines a peak of work intensity and calculates a pulse rate at the peak of work intensity. The pulse rate and risk factor “R” are input to a second pulse rate threshold calculation module 254. The second pulse rate threshold 15, calculation module 254 calculates a second pulse rate threshold as follows.

Second pulse rate threshold=120−α3R+β3

where β3 is a fixed value determined according to a hot environment.

α1, α2, and α3 are predetermined coefficients.

A comparison module 256 compares the pulse rate and the second pulse rate threshold. A result of the comparison is input to the determination part 260.

Each of the comparison modules 236, 244, and 256 outputs a result of comparison with the threshold in a binary manner. When a target to be compared is higher than the threshold, “1” is output. The determination module 260 calculates a logical sum of the outputs of the comparison modules 244 and 256, and determines the presence or absence of heat stress periodically, for example, every one minute, by a logical product of the logical sum and the output of the comparison module 236.

According to the sixth embodiment, a pulse rate relates to the determination of the presence or absence of heat stress. Like the heart rate measured by the electrocardiograph 24 of the first to fifth embodiments, the pulse rate measured by the pulse wave sensor 202 is likely to decrease in its reliability if a body moves. It is therefore useful to obtain a result of determination of the presence or absence of heat stress and its reliability together, cause the display/communication control module 126 to display both of them on the display 70 of the mobile terminal 12, and supply them to the server 14 via the wireless communication device 62.

The physical stress indices other than WBGT may include a heat stress risk level or sensor data that take into consideration age, weight, and the like. The heat stress risk level may be derived from worker's ambient temperature, skin temperature, CO₂ concentration, a heart rate, and acceleration. The sensor data may include exercise intensity (METs), steps, a heart rate, worker's ambient temperature, and relative humidity. Reliability may also be calculated for these physical stress indices other than WBGT.

[Modification to Embodiments]

According to the foregoing embodiments, the reliability of LF/HF is calculated as the reliability of a physical stress index. The LF/HF is calculated by analyzing the frequency of a heartbeat interval. However, information based on which the reliability is calculated is not limited to the LF/HF calculated by analyzing the frequency of a heartbeat interval. The LF/HF may be calculated using a pulse interval in place of the heartbeat interval. The physical stress index is not limited to the LF/HF, but may be the LF only, the HF only, the VLF, the total power, the “HRT”, the “Mean”, the “SDNN”, the “RMSSD”, the “NN50”, the “pNN 50”, and the like. The reliability of a heat index such as WBGT index may also be calculated.

The sensor device 10 is not limited to a wearable device, but may be any type of sensor as long as its electrodes are in contact with the body of a user.

The sensor that acquires a heartbeat interval is not limited to the electrocardiograph, but may be configured by a wearable measurement device or a wearable sensor having the same function, a camera, a radar transmitter, or the like. A camera, a radar transmitter, or the like may also configure the pulse wave sensor.

Furthermore, the information based on which the reliability is calculated is not limited to the stress index but may be any biological information. The biological information may include information indicative of the state of a living body, information that affects the state of a living body, information obtained by calculating the information indicative of the state of a living body, and information obtained by calculating the information indicative of the state of a living body. The information indicative of the state of a living body may include a height, a weight, a BMI, a degree of obesity, an abdominal girth, a body-fat percentage, a blood pressure, an eyesight, a hearing, urinalysis results, blood test results, an electrocardiogram, an electromyogram, a sweat quantity, a skin moisture, skin stains, skin wrinkles, genomic information, and the like. The information that affects the state of a living body may include a discomfort index due to heat, a discomfort index due to cold, feeling, expression, am age, a facial image, and the like.

Means for acquiring the biological information may include an equipment to acquire information, a device such as a sensor to acquire information, sending and answering questionnaires, and the like.

As the biological information, information acquired by acquisition means such as a sensor may be used as it is or information obtained by processing the acquired information appropriately may be used. For example, the height and the weight are measured, a BMI is calculated from the measured height and weight, and the reliability of the BMI may be calculated. The biological information acquisition means may receive the BMI that has already been processed.

The reliability is acquired using information that can affect data accuracy of the biological information or information that is to be an index for determining the data accuracy. This information may be a single information item (heart rate) as in the first embodiment or a plurality of information items (heart rate, body motion, sweat quantity, and contact pressure) as in the embodiments other than the first embodiment. Information that can affect data accuracy of the heartbeat interval or that is to be an index for determining the accuracy may include information regarding the body, such as a BMI, a degree of obesity, a body-fat percentage, a sweat quantity, a skin moisture, a skin dirt, skin stains, skin wrinkles, and genomic information, environmental information regarding a temperature, a humidity, an atmospheric pressure and the like, information regarding relationship between a measurement device and a body, such as the state of contact, a contact pressure and a distance between the measurement device and the skin, information regarding a discomfort index due to heat, a discomfort index due to cold, feeling, expression, an age, a facial image, and the like. The greater the subcutaneous fat, the higher the impedance, and the higher the body-fat percentage, the lower the reliability.

Means for acquiring the above biological information may include an equipment to acquire biological information, a sensor, a camera, sending and answering questionnaires, and the like.

As the embodiments, a system in which the sensor device 10, the mobile terminal 12, and the server 14 cooperate with each other, has been described. An embodiment in which the system includes the sensor device 10 alone and an embodiment of the system excluding the server 14 and including the sensor device 10 and the mobile terminal 12 which cooperate with each other can be achieved. The former embodiment is achieved by storing the stress estimation program 66b in the flash memory 34 of the sensor device 10 and executing the stress estimation program 66b loaded in the memory 30. The latter embodiment is achieved by sharing the functions of the server 14 between the mobile terminal 12 and the sensor device 10.

In the foregoing embodiments, computer programs can achieve the processing. Accordingly, the same advantageous effects as those of the embodiments can easily be obtained even through a computer-readable storage medium storing the computer programs or by installing the computer programs in a computer through a communication medium and executing the computer programs.

While certain embodiments have been described, these embodiments have been presented by way of example only, and are not intended to limit the scope of the inventions. Indeed, the novel embodiments described herein may be embodied in a variety of other forms; furthermore, various omissions, substitutions, and changes in the form of the embodiments described herein may be made without departing from the spirit of the inventions. The accompanying claims and their equivalents are intended to cover such forms or modifications as would fall within the scope and spirit of the inventions.

What is claimed is:

1. An electronic apparatus comprising:
one or more processors configured to
acquire first information related to reliability of biological information;
calculate reliability information of the biological information based on the first information; and
associate the reliability information with the biological information.

2. The electronic apparatus of claim 1, wherein the first information comprises information indicative of at least one of a body motion, a heart rate, a pulse rate, a BMI, a degree of obesity, a body-fat percentage, an amount of sweat, a skin moisture, a skin dirt, skin stains, skin wrinkles, genomic information, a temperature, a dry bulb temperature, a wet bulb temperature, a globe temperature, a humidity, an atmospheric pressure, a state of contact between a living body and a sensor which senses the biological information, a pressure of contact between the sensor and the living body, a distance between the sensor and the living body, a discomfort index due to heat, a discomfort index due to cold, feeling, expression, an age, and a facial image.

3. The electronic apparatus of claim 1, wherein the biological information includes information indicative of at least one of a heart rate, a pulse rate, a height, a weight, a BMI, a degree of obesity, an abdominal girth, a body-fat percentage, a blood pressure, an eyesight, a hearing, urinalysis results, blood test results, an electrocardiogram, an electromyogram, an amount of sweat, a skin moisture, skin stains, skin wrinkles, genomic information, a discomfort index due to heat, a discomfort index due to cold, feeling, expression, an age, a facial image, a temperature, a dry bulb temperature, a wet bulb temperature, a globe temperature.

4. The electronic apparatus of claim 1, further comprising:
a sensor configured to sense a state of a living body, and wherein the biological information comprises a stress index calculated based on an output of the sensor.

5. The electronic apparatus of claim 1, wherein the one or more processors are configured to input the biological information.

6. The electronic apparatus of claim 1, further comprising:
a sensor configured to sense a state of a living body, and wherein the one or more processors are configured to restore a lost part of an output of the sensor based on the output of the sensor when the reliability of the biological information is higher than a first reliability.

7. The electronic apparatus of claim 1, wherein the one or more processors are configured to display an icon or a text in association with the biological information, the icon or the text representing the reliability of the biological information, levels of the reliability in a plurality of stages, or meanings of the reliability.

8. The electronic apparatus of claim 1, wherein the one or more processors are configured to transmit the reliability information and the biological information to an external device.

9. An electronic apparatus comprising:
one or more processors configured to
acquire first information items related to reliability of biological information;
calculate reliability information items of the biological information based on the first information items; and
determine a cause for which the reliability of the biological information is equal to or lower than a first reliability when the reliability of the biological information is equal to or lower than the first reliability.

10. The electronic apparatus of claim 9, wherein the one or more processors are configured to display countermeasures to remove the cause.

11. The electronic apparatus of claim 9, wherein the one or more processors are configured to determine that the reliability of the biological information is not equal to or lower than the first reliability when at least one of the reliability information items is not equal to or lower than the first reliability.

12. The electronic apparatus of claim 9, wherein the one or more processors are configured to display each of the reliability information items or one integrated reliability information item obtained by combining the reliability information items.

13. The electronic apparatus of claim 9, wherein the first information comprises information indicative of at least one of a body motion, a heart rate, a pulse rate, a BMI, a degree of obesity, a body-fat percentage, an amount of sweat, a skin moisture, a skin dirt, skin stains, skin wrinkles, genomic information, a temperature, a dry bulb temperature, a wet bulb temperature, a globe temperature, a humidity, an atmospheric pressure, a state of contact between a living body and a sensor which senses the biological information, a pressure of contact between the sensor and the living body, a distance between the sensor and the living body, a discomfort index due to heat, a discomfort index due to cold, feeling, expression, an age, and a facial image.

14. The electronic apparatus of claim 9, wherein the biological information includes information indicative of at least one of a heart rate, a pulse rate, a height, a weight, a BMI, a degree of obesity, an abdominal girth, a body-fat percentage, a blood pressure, an eyesight, a hearing, urinalysis results, blood test results, an electrocardiogram, an electromyogram, an amount of sweat, a skin moisture, skin stains, skin wrinkles, genomic information, a discomfort index due to heat, a discomfort index due to cold, feeling, expression, an age, a facial image, a temperature, a dry bulb temperature, a wet bulb temperature, a globe temperature.

15. The electronic apparatus of claim 9, further comprising:
a sensor configured to sense a state of a living body, and wherein the biological information comprises a stress index calculated based on an output of the sensor.

16. The electronic apparatus of claim 9, wherein the one or more processors are configured to input the biological information.

17. The electronic apparatus of claim 9, wherein the one or more processors are configured to display an icon or a text in association with the biological information, the icon or the text representing the reliability of the biological information, levels of the reliability in a plurality of stages, or meanings of the reliability.

18. A method comprising:
acquiring first information related to reliability of biological information;
calculating reliability information of the biological information based on the first information; and
associating the reliability information with the biological information.

19. The method of claim 18, further comprising:
sensing, by a sensor, a state of a living body, and wherein the biological information comprises a stress index calculated based on an output of the sensor.

20. The method of claim 18, further comprising:
displaying an icon or a text in association with the biological information, the icon or the text representing the reliability of the biological information, levels of the reliability in a plurality of stages, or meanings of the reliability.
